(19) United States
(12) Patent Application Publication
(10) Pub. No.: US 2018/0338904 A1
(43) Pub. Date: Nov. 29, 2018

(54) SINGLE CHANNEL, MULTIPLE DRUG DELIVERY DEVICE AND METHODS

(71) Applicant: Inguran, LLC, Navasota, TX (US)

(72) Inventors: Scott A. Uhland, San Jose, CA (US); Ramkumar Abhishek, Mountain View, CA (US); Eric Peeters, Mountain View, CA (US); Timothy J. Curley, San Carlos, CA (US)

(73) Assignee: Inguran, LLC, Navasota, TX (US)

(21) Appl. No.: 15/996,349

(22) Filed: Jun. 1, 2018

Related U.S. Application Data
(62) Division of application No. 13/629,124, filed on Sep. 27, 2012.

(51) Int. Cl. A61K 9/00; A61M 31/00

(52) U.S. Cl. CPC A61K 9/0004; A61M 31/002; A61K 9/0034

(57) ABSTRACT

Devices and methods are provided for drug delivery. The device may include a housing configured for intralumenal deployment into a human or animal subject and a reservoir contained within the housing and having an actuation end and a release end. The release end may include at least one outlet. A first drug formulation and a second drug formulation may be disposed within the reservoir and adjacent to each other and immiscible, or separated from each other by a first barrier. The device may also include a plug within the reservoir at the actuation end, the plug being movable toward the release end to drive the first and second drug formulations out of the reservoir. The device may also include an actuation system operably connected to the actuation end of the reservoir and configured to drive the plug toward the release end and release the drug formulations from the reservoir.

### PRIORITY

This application is a Divisional of U.S. application Ser. No. 13/629,124, filed Sep. 27, 2012.

### FIELD

The present disclosure is generally in the field of drug delivery devices and methods, and more particularly to devices and methods for the transmucosal delivery of multiple drugs to human or animal subjects.

### BACKGROUND

Controlled delivery of multiple drugs from a single device is an area of interest because of the potential of delivering a series of drugs in a treatment regimen in a specific release profile. For example, current fixed time artificial insemination (FTAI) treatments for cattle require the administration of multiple drugs at specific times. These treatments result in significant time spent driving, herding, and chuting the cattle, cause stress and increased cortisol levels in the subjects, and require multiple drug delivery devices and precise drug administration timing.

Transmucosal drug delivery is an area of interest because of the potential of delivering systemically-acting drugs with a high relative bioavailability by avoiding first-pass metabolism effects, the potential of locally delivering therapeutic agents to a site of interest, and the convenience of application routes. Some of the possible sites for transmucosal drug delivery include the buccal, nasal, vaginal, and rectal administration routes.

Accordingly, it would be desirable to provide improved devices and methods to transmucosally administer multiple drug formulations from a single device to human or animal subjects.

### SUMMARY

In one aspect, a device for drug delivery is provided, which includes a housing configured for intralumenal deployment into a human or animal subject and a reservoir, which has an actuation end and a release end, contained within the housing. The release end includes at least one outlet. A first drug formulation and a second drug formulation are disposed within the reservoir. A plug is also included within the reservoir at the actuation end and is movable toward the release end to drive the first and second drug formulations out of the reservoir. The device also includes an actuation system operably connected to the actuation end of the reservoir and configured to drive the plug toward the release end such that the first drug formulation is released from the reservoir before the second drug formulation is released from the reservoir. The first and second drug formulations are either adjacent to each other and immiscible, or separated from each other by a first fluid barrier.

In another aspect, the first drug formulation and the second drug formulation are disposed within the reservoir and separated by a first barrier. A plug, which is positioned at the actuation end within the reservoir, is movable toward the release end to drive the first and second drug formulations out of the reservoir. An actuation system is operably connected to the actuation end of the reservoir and configured to drive the plug toward the release end such that the first drug formulation is released from the reservoir before the second drug formulation is released from the reservoir. A barrier retention chamber is connected to the release end of the reservoir and configured to receive and retain the first barrier before the release of the second drug formulation.

In yet another aspect, a method of drug delivery is provided, which includes deploying a drug delivery device into a mucosal lumen of a human or animal subject, actuating an actuation system to drive the first drug formulation out of the reservoir, and thereafter actuating the actuation system to drive the second drug formulation out of the reservoir, wherein the drug delivery device includes a reservoir containing a first drug formulation and a second drug formulation, which are either adjacent to each other and immiscible from one another, or separated by a first fluid barrier.

In still another aspect, a method is provided for fixed time artificial insemination. The method includes deploying a drug delivery device into a vaginal lumen of an animal subject; actuating an actuation system to release a first drug formulation out of a reservoir of the device at a first time, to release a second drug formulation out of the reservoir at a second time, to release a third drug formulation out of the reservoir at a third time; and artificially inseminating the animal subject at a fourth time following the first, second, and third times. The drug delivery device includes a reservoir containing a first drug formulation comprising a gonadotropin-releasing hormone, a second drug formulation comprising a prostaglandin, and a third drug formulation comprising a gonadotropin-releasing hormone.

### BRIEF DESCRIPTION OF THE DRAWINGS

FIG. 1 is a cross-sectional view, illustrating one embodiment of a drug delivery device having two drug formulations separated by a first barrier, in a tissue lumen.

FIG. 2 is a cross-sectional view, illustrating one embodiment of a drug delivery device having two drug formulations that are adjacent to each other and immiscible, in a tissue lumen.

FIG. 3 is a cross-sectional view, illustrating one embodiment of a drug delivery device having four drug formulations that are separated by multiple barriers, in a tissue lumen.

FIG. 4A is a cross-sectional view, illustrating one embodiment of a drug delivery device prior to actuation.

FIG. 4B is a cross-sectional view, illustrating the drug delivery device of FIG. 4A upon actuation.

FIG. 4C is a cross-sectional view, illustrating the drug delivery device of FIG. 4A at a first later time following actuation.

FIG. 4D is a cross-sectional view, illustrating the drug delivery device of FIG. 4A at a second later time following actuation.

FIG. 4E is a cross-sectional view, illustrating the drug delivery device of FIG. 4A at a third later time following actuation.

FIG. 4F is a cross-sectional view, illustrating the drug delivery device of FIG. 4A at a fourth later time following actuation.

FIG. 5A is a perspective view, illustrating one embodiment of a drug delivery device.

FIG. 5B is a partially exploded plan view, illustrating the drug delivery device of FIG. 5A.

FIG. 6A is a partially exploded cross-sectional view, illustrating one embodiment of an electrolytic cell for use in an actuation system of one embodiment of a drug delivery device.

FIG. 6B is a perspective view, illustrating the electrolytic cell of FIG. 6A.

### DETAILED DESCRIPTION

The devices and methods described herein provide for the storage and controlled delivery of multiple drug formulations. The devices are advantageously configured to separately store multiple drug formulations, thereby minimizing the risk of contamination, and to precisely dispense the drug formulations according to a specific release timing profile. These devices and methods desirably provides for a single device to deliver a series of drug doses to a human or animal subject at prescribed times. The devices and methods can significantly increase the accuracy and efficiency of delivering multiple agents to subjects, which can be particularly advantageous, for example, in large scale animal husbandry operations, such as artificial insemination.

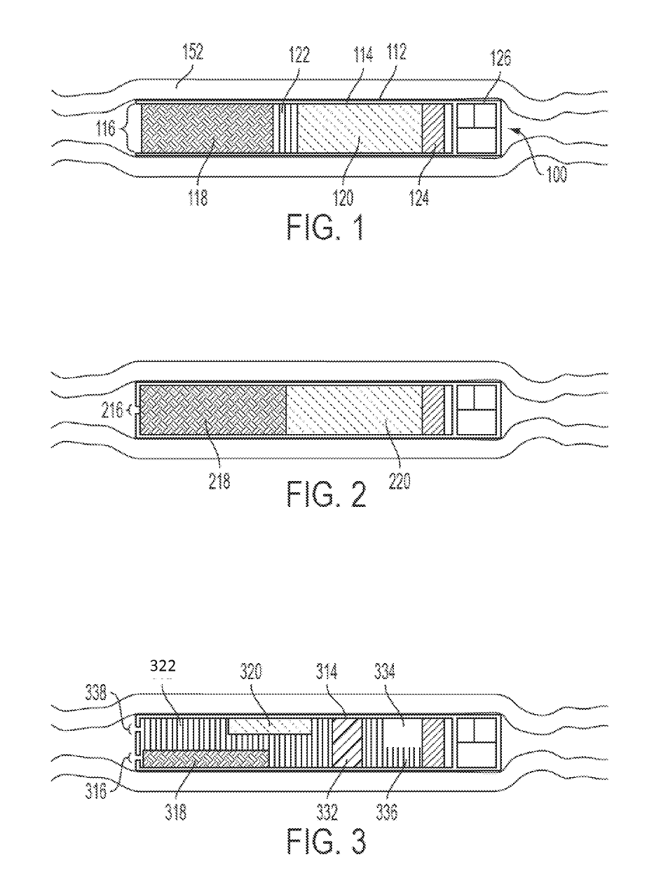

In one aspect, a device for drug delivery is provided. As shown in FIG. 1, the device 100 includes a housing 112 configured for intralumenal deployment into a human or animal (e.g., mammalian) subject. The term “intralumenal,” as used herein, refers to placement within a body cavity, channel, tube, or the like, having a mucosal wall. The term includes, but is not limited to, sites in the reproductive tract, such as intravaginal, cervical, or intrauterine, and the gastrointestinal tract, such as intrarectal.

The device 100 includes a reservoir 114 contained within the housing 112. The reservoir 114 has an actuation end portion and a release end portion. The release end portion includes at least one outlet 116, as shown in FIG. 1. In one embodiment, first and second drug formulations 118, 120 are disposed within the reservoir 114 and are separated from each other by a first barrier 122. In a particular embodiment, the first barrier sealingly engages with, and can slide with respect to, the inner wall of the reservoir. In another embodiment, as shown in FIG. 2, first and second drug formulations 218, 220 are adjacent to each other and are substantially immiscible in one another. As shown in FIG. 1, the device 100 also includes a plug 124 within the reservoir 114 that is movable from the actuation end toward the release end to drive the first and second drug formulations 118, 120 out of the reservoir 114, thereby delivering the drug formulations to the lumenal tissue adjacent the release end of the reservoir. In a particular embodiment, the plug also sealingly engages with, and slides with respect to, the inner wall of the reservoir. The plug may function as a piston. An actuation system 126 is operably connected to the actuation end of the reservoir 114 and is configured to drive the plug 124 toward the release end such that the first drug formulation 118 is released from the reservoir 114 before the second drug formulation 120 is released from the reservoir 114.

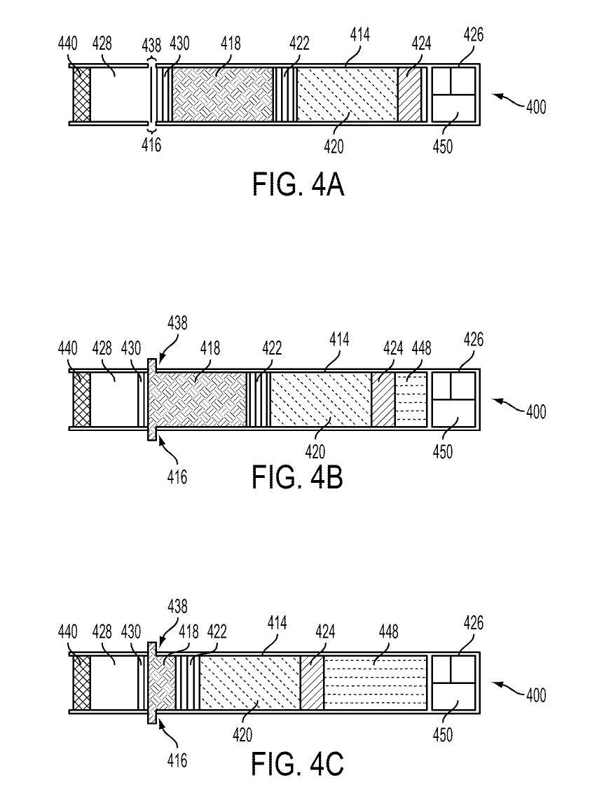

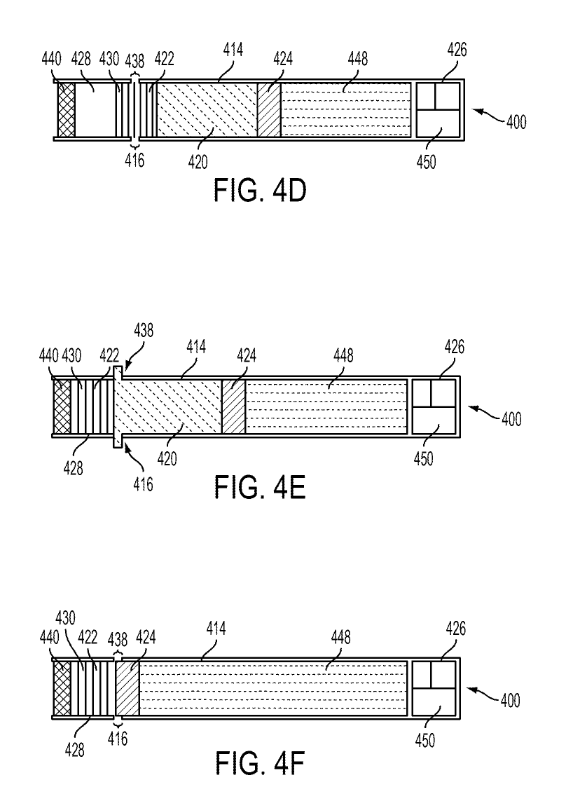

As shown in FIGS. 4A-4F, the device 400 may further include a barrier retention chamber 428 connected to the release end of the reservoir 414 and configured to receive and retain the first barrier 422 before the release of the second drug formulation 420. In one embodiment, as shown in FIGS. 4A-4F, the device includes a second barrier 430 positioned between the barrier retention chamber 428 and the first drug formulation 418, such that the first drug formulation 418 cannot enter the barrier retention chamber 428.

In another aspect, a method of drug delivery is provided. The method may include (i) deploying a drug delivery device having a reservoir containing first and second drug formulations into a mucosal lumen of a human or animal subject, (ii) actuating an actuation system to drive the first drug formulation out of the reservoir; and thereafter (iii) actuating the actuation system to drive the second drug formulation out of the reservoir.

Various embodiments and features of the drug delivery devices and methods are described in greater detail hereinafter.

### Housing

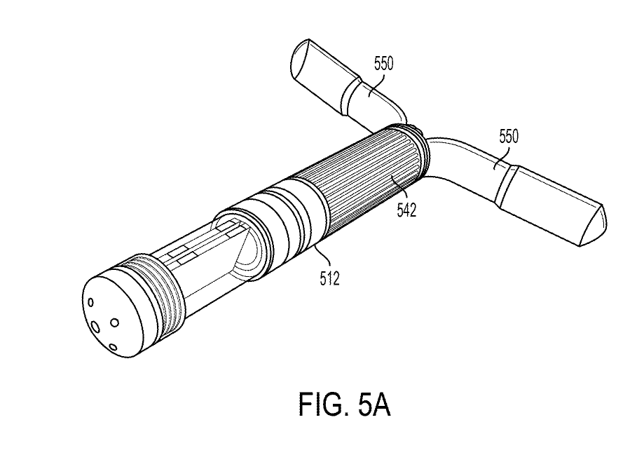

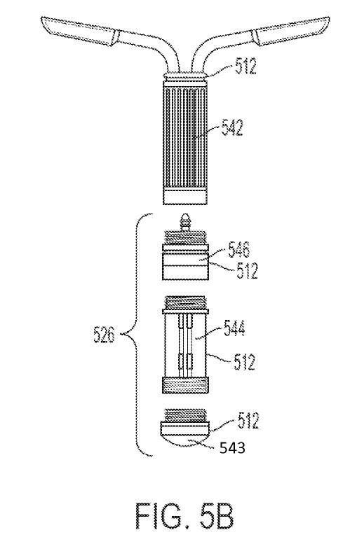

The device includes a housing generally configured to facilitate deployment of the drug delivery device within a lumen of a human or animal subject. The housing configuration is based upon the particular lumenal site and human or animal anatomical considerations, for deployment with minimal discomfort to the subject. In certain embodiments, the device may be placed within the lumen by insertion into the lumen via an exterior body orifice. Accordingly, in certain embodiments, the housing is shaped and dimensioned to allow insertion and placement, i.e., deployment, of the device within the intended lumen via the exterior body orifice. For example, the housing may be shaped and dimensioned for vaginal, cervical, uterine, or rectal insertion and placement. As shown in FIGS. 5A-5B, the housing 512 may include an elongated, substantially cylindrical portion having wing-like portions, or arms, 550 extending therefrom. For example, this configuration may be appropriate for vaginal device deployment in livestock, such as cattle, sheep, etc.

The materials of construction, size, shape, surface features, and other characteristics of the housing are configured such that the device can be deployed into the lumen, retained securely in the lumen during operation of the device, and retrieved from the lumen following operation of the device or when otherwise desired to be removed. For example, the device may be removed between the delivery of individual drug formulations, following the delivery of several drug formulations, or following the completion of a course of treatment of multiple drug formulations. The device may be deployed until the drug formulation payload is depleted.

The housing may be formed of any biocompatible material. Moreover, the housing material may be resistant to degradation in the mucosal environment of the lumen. Examples of suitable housing materials include stainless steel, titanium, and certain polymers, ceramics or composited of one of these materials. The housing material may include a coating to enhance biocompatibility and/or operation of the device.

### Reservoir and Contents

At least one reservoir is located within the housing. The reservoir has an actuation end operably connected to an actuation system, as well as a release end which includes at least one outlet for releasing the drug formulations to the lumenal tissue. For example, the reservoir may be defined by an inner surface of an elongated annular tube. One end of the reservoir may be connected to the actuation end and the opposite end may include an outlet such as an aperture or nozzle. Multiple outlets may also be provided. The reservoir may also have a shape similar to that of the housing and be configured such that it occupies a majority of the volume of the housing. In certain embodiments, the reservoir is elongated and has a circular cross-sectional shape. Other cross-sectional shapes are also envisioned.

In one embodiment, the reservoir contains first and second drug formulations. The device may include more than two drug formulations within the reservoir. For example, the reservoir may contain three or four drug formulations. In particular embodiments, the multiple drugs are ones selected to work in concert, but beneficially are administered in series, for example in a separated or overlapping schedule.

In certain embodiments, as shown in FIG. 1, the first and second drug formulations 118, 120 are separated from each other by a first barrier 122. The first barrier may be a fluid barrier or a solid barrier. For example, the fluid barrier may be an inert gel. Inert gels may include polyvinyl alcohol (PVA), poly(ethylene glycol) (PEG), hyaluronic acid (HA), cellulose, polyvinyl pyrrolidone (PVP), polyacrylic acid (PAA), polyethylene oxide (PEO), poly(p-phenylene oxide) (PPO), polyacrylamides, N-(2-hydroxypropyl) methacrylamide (HPMA), divinyl ether-maleic anhydride (DIVEMA), poly(2-alkyl-2-oxazolines), polyphosphates, polyphosphazenes, xanthan gum, polysaccharides, chitosan derivatives, carrageenan, cellulose ethers, starches, formulations of silicone elastomers such as polydimethylsiloxane (PDMS), or combinations thereof. For example, the solid barrier may be a biocompatible plunger. Solid barriers may include higher molecular weight polyvinyl alcohol (PVA), poly(ethylene glycol) (PEG), hyaluronic acid (HA), cellulose, polyvinyl pyrrolidone (PVP), polyacrylic acid (PAA), polyethylene oxide (PEO), poly(p-phenylene oxide) (PPO), polyacrylamides, N-(2-hydroxypropyl) methacrylamide (HPMA), divinyl ether-maleic anhydride (DIVEMA), poly(2-alkyl-2-oxazolines), polyphosphates, polyphosphazenes, xanthan gum, polysaccharides, chitosan derivatives, carrageenan, cellulose ethers, starches, formulations of silicone elastomers such as polydimethylsiloxane (PDMS), or combinations thereof.

FIG. 2 shows a configuration of the device of FIG. 1 in which the first and second drug formulations 218, 220 are adjacent to each other and are essentially immiscible in one another such that a first barrier is unnecessary. As used herein, the term “immiscible” refers to the first and second drug formulations being substantially incapable of being mixed to form a homogenous substance. In one embodiment, the first and second drug formulations are either adjacent to each other and immiscible or are separated from each other by a first fluid barrier. In one embodiment, the device may include a third drug formulation that is either adjacent to and immiscible with the second drug formulation, or that is separated from the second drug formulation by a second fluid barrier. FIG. 3 shows a configuration of the device of FIG. 1 in which four drug formulations 318, 320, 332, 334 are disposed within the reservoir and are separated by multiple fluid barriers 322, 336.

The reservoir also includes a plug which is movable from the actuation end of the reservoir toward the release end of the reservoir. The plug is configured to drive the drug formulations and any barriers therebetween out of the reservoir. The plug generally is positioned between the actuation system and the drug formulations. The plug may include a fluid layer or a solid barrier. For example, the fluid layer may include an inert gel. Alternatively, the plug may include a biocompatible plunger.

### Release Structure

In embodiments, the device is configured to deliver the drug formulations to the mucosal tissue of the lumen in which the device is deployed. The drug formulations are released from at least one outlet in at the release end portion of the reservoir, toward which the plug drives the drug formulations. The release end portion of the reservoir may be configured to release the drug formulations from the device axially, radially, or a combination thereof. In certain embodiments, the device includes a structure interposed between the at least one outlet and the tissue lumen. Such structures may function to redirect or spread the drug formulation across a greater area of the tissue lumen, and/or may function to control release kinetics of the drug. For example, the device may include a porous membrane configured to diffuse the drug formulations released from the at least one outlet to the lumenal tissue.

FIGS. 2-3 show configurations of the device of FIG. 1 in which an end cap is provided at the release end. In one embodiment, as shown in FIG. 2, the end cap has a single outlet 216 therein. In another embodiment, as shown in FIG. 3, the end cap has two outlets 316, 338 therein. In other embodiments, the end cap may have more than two outlets. As shown in FIG. 3, the reservoir 314 may include an end cap having a first outlet 316 and a second outlet 338 therein, with the first and second outlets 316, 338 positioned such that the first drug formulation 318 is released only out of the first outlet 316 and the second drug formulation 320 is released only out of the second outlet.

In one embodiment, a ring of outlets is provided at the release end of the reservoir. For example, a ring of outlets may be radially positioned at the release end portion of a cylindrical reservoir. As shown in FIGS. 4A-4F, outlets 416, 438 are radially positioned at the release end of reservoir 414.

In certain embodiments, as shown in FIGS. 4A-4F, the device 400 includes a barrier retention chamber 428 connected to the release end of the reservoir 414 and configured to receive and retain the first barrier 422 before the release of the second drug formulation 420. The barrier retention chamber may be sized to receive and retain the one or more barriers used to separate the drug formulations in the reservoir. For example, as shown in FIGS. 4A-4F, the barrier retention chamber 428 may be sized to receive both the first barrier 422 and the second barrier 430. The barrier retention chamber 428 may include a hydrophobic vent 440 to allow fluid to be displaced from the barrier retention chamber as it becomes filled with the first, second, and any other barriers. For example, the vent may include a PTFE based membrane, a polypropylene membrane, or a PTFE-coated membrane having an open pore structure. Micro-one way valves, umbrella valves, and duck bill valves may also be used as vents. Such valves may be constructed of silicone or other materials.

In certain embodiments, as shown in FIGS. 5A-5B, the housing 512 includes a porous membrane sidewall 542 in fluid communication with the at least one outlet of the release end of the reservoir. The porous membrane sidewall may be configured to control diffusion of the first and second drug formulations released from the reservoir. For example, the porous membrane sidewall may diffuse the drug formulations over a region of the tissue membrane adjacent thereto. For example, the porous membrane sidewall may include a polycarbonate, polypropylene, PTFE, or polyethylene membrane, or any combination of laminates thereof. For example, the porous sidewall membrane may have a pore size from about 0.2 μm to about 25 μm. For example, the porous membrane sidewall may be as described in U.S. patent application Ser. No. 13/629,159, entitled “Multiple Reservoir Drug Delivery Device and Methods,” which is filed concurrently herewith and the disclosure of which is incorporated herein by reference in its entirety.

### Actuation System

The device includes an actuation system which is operably connected to the actuation end of the reservoir and is configured to drive the plug toward the release end to release the drug formulations from the reservoir. Generally, the actuation system is configured to drive the plug via a positive displacement process. The term “positive displacement,” as used herein, refers to any process whereby the drug formulations are dispensed from the drug delivery device under force provided by the plug within the reservoir. Accordingly, the term does not refer to the passive, chemical diffusion of the drug formulations out of the reservoir, although passive diffusion may contribute to release of the drug formulations from the porous membrane. As shown in FIGS. 5A-5B, the actuation system 526 may include a power source 543, a microcontroller 544, and an actuation mechanism 546.

The power source may be any source of mechanical, electrical power or electromechanical power. The power source may include one or more batteries or fuel cells.

The microcontroller may be configured to control the actuation system of the device, and thereby control the timing of release of the drug formulations. For example, the microcontroller may selectively transmit electrical or mechanical power to the actuation mechanism, advancing the plug through the reservoir and dispensing the drug formulations. The microcontroller may be configured to control the timing of delivery of the drug formulations by applying the necessary electrical potentials to the actuation mechanism. The controller may be programmable or it may be pre-programmed to deliver the drug formulations in accordance with a prescribed release schedule.

The actuation mechanism may include fluid-volume displacement, mechanical displacement, osmotic swelling displacement, electrostatically-induced compression, piezoelectric actuation, thermally/magnetically induced phase transformation, or combinations thereof, to drive the plug via positive displacement.

In certain embodiments, as shown in FIGS. 4B-4F, the actuation system 426 is configured to generate a displacement fluid 448 in operable communication with the plug 424 to drive the plug, and the drug formulations 418, 420, toward the release end by a positive displacement process. For example, the actuation system 426 may include an electrolytic cell 450 having a cathode and an anode which contact water or an aqueous solution to generate a gas 448, such as oxygen, in contact with the plug 424.

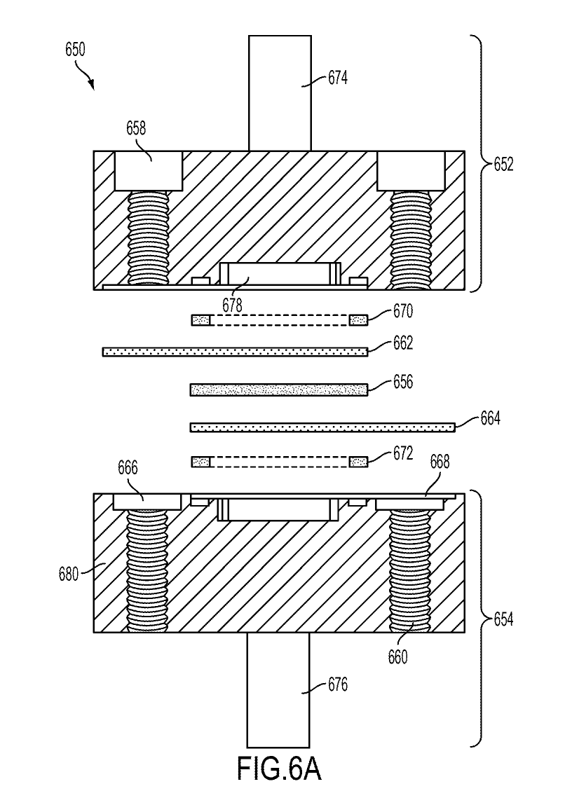

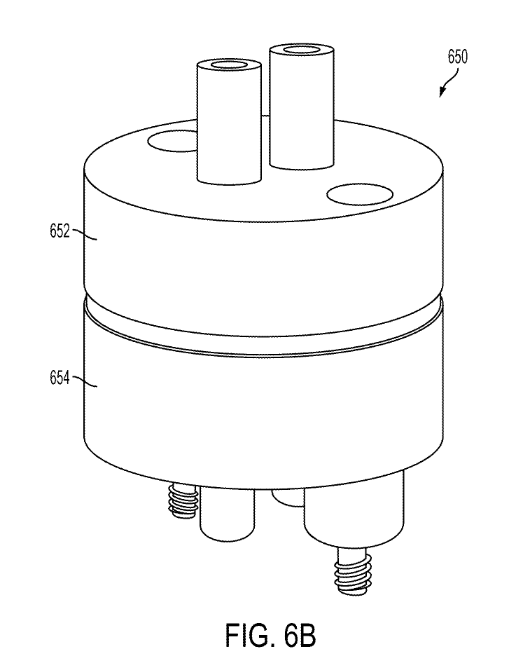

FIGS. 6A-6B show one embodiment of an electrolytic cell 650. The cell 650 includes cathode assembly 652 and anode assembly 654, which are assembled to be in intimate contact. The intimate contact may be achieved by chemical or thermal surface modification (including but not limited to epoxies and adhesives), mechanical compression (including but not limited to screw-based torque application), welding, soldering. In one embodiment, the units are assembled to be in intimate contact by means of chemical surface modification including but not limited to an epoxy-based seal. The electrode assembly units can be made of a variety of materials including but not limited to plastics, metals, and polymers. In one embodiment, the units are made of a high volume manufacturing-compliant plastic, such as polypropylene. The electrodes may be made of a variety of materials including metallized substrates, conductive and/or metallized polymers. In one embodiment, the electrodes are made of porous planar metallized polymer substrates such as metallized polyester or metallized PEN.

Cathode assembly 652 and anode assembly 654 are arranged to be in contact with active component 656 on either side. The electrodes may be permeable to provide access to the active component, for example electrodes may include fabricated and/or naturally occurring macroscopic or microscopic pores. Gaseous products, such as H₂ and O₂, may be generated when energy is applied to the active component, including but not limited to electrical energy and thermal energy. For example, active component 656 may be a sulfonated tetrafluoroethylene based fluoropolymer-copolymer which is highly selective and permeable to water, such as Nafion. When electrical energy is applied to a hydrated Nafion layer H₂ and O₂ gases are generated by methods including but not limited to electrolysis of water. Other active components such as ionic solutions, hydrogels, H₂O₂, and other fluids that can be electrolyzed to generate gaseous products may also be used.

Electrical contact to the cathode and anode assemblies 652, 654 is achieved via screws 658, 660, perforated electrodes 662, 664 and nuts 666, 668. The components are arranged such that the screws 658, 660 are used for both fastening and providing isolated electrode contacts to the anode and cathode assembles 652, 654. A low-resistance and uniform electrical contact along the surface of the active component 656 may be achieved by using planar perforated electrodes 662, 664 having holes therein to allow the screws to pass through. The nuts 666, 668 serve as the electrical contact between the perforated electrodes and the screws. Other forms of electrical contact to the electrodes may also be used, such as flex-cables, for example metal on a flexible polymer substrate, printed circuit boards, screw-based contact, and soldering wires.

Gaseous isolation between the anode and cathode assemblies 652, 654 is achieved using compression-based gasket sealing with O-rings 670, 672, which are compressed by fastening the two assemblies 652, 654 together using screws 658, 660. Other methods of sealing such as epoxy and metallic weld/solder may also be used. Gas collection is achieved by directing the gas generated at the active component through conduits in the electrode assembly units into structures such as a nozzle or chamber. For example, at least one outlet port 674, 676 is provided at each of the anode and cathode assemblies 652, 654 for gas collection of H₂ and O₂, respectively, and to provide access to the active component between the electrodes. The outlet ports 674, 676 also provide water perfusion to hydrate the Nafion layer 656.

In order to ensure uniform hydration and gas collection from the active layer 656, the anode and cathode assemblies 652, 654 include flow-fields 678, 680 which help maximize the amount of gas generated and collected from the active layer. The flow-field pattern can be any shape or pattern configured to maximize the accessible area of the active layer 656, and thereby maximize the amount of gas produced and collected. For example, the flow-fields may include a meandering conduit. Gas-permeable substrates may also be used to maximize gas generation and collection.

FIG. 6B shows an assembled electrolytic cell 650. The call may have a diameter of about 25.5 mm and a height of about 19 mm. Other dimensions also are envisioned. The electrical connections to the anode and cathode are made on one side, namely the anode assembly 654 to ensure complete gaseous isolation at the cathode assembly 652.

In certain embodiments, a multi-actuator assembly can be made using multiple electrolytic cells spatially arranged within a single structure to allow for localized and isolated generation of gases at specified locations. The cells can be pre-assembled or assembled together in order to have intimate contact by methods such as chemical or thermal surface modification (including but not limited to epoxy and adhesives), mechanical compression (including but not limited to screw-based torque application), welding, and soldering. In one embodiment, the individual cells share the same active component. Activation may be achieved using separate electrode pairs for each cell or by using a shared electrode or electrodes. The gases generated may be collected and mixed between cells to produce a higher volume of gas at a particular location in the structure.

In one embodiment, a channel is provided in the housing to allow aqueous secretions from the mucosal tissue of the lumen to contact the cathode and anode. In one embodiment, water or an aqueous solution is contained on-board the device. For example, the actuation system may include a reservoir containing an electrolytic solution, for example an ionic solution such as sodium nitrite. In one embodiment, the actuation system includes a reservoir containing deionized water and a solid electrolyte contacting the surfaces of the cathode and anode.

An electrical potential of about 1.0 V or greater may be applied to the electrodes of the electrolytic cell to generate oxygen at the anode. The reaction at the anode is described by EQ. 1. In the water, at the negatively charged cathode, a reduction reaction takes place, with electrons from the cathode being given to the hydrogen cations to form hydrogen gas as shown in EQ. 2. The pressure exerted by the generated oxygen and hydrogen causes the plug to advance through the reservoir, thereby causing the drug formulations to be released at the release end into the lumen. The production of oxygen and hydrogen may be controlled by the power source and a microcontroller that is programmed to supply an electrical potential to the cathode and anode at a selected time.

2H₂O (l)→O₂(g)+4H⁺(aq)+4e⁻ EQ. 1

2H⁺(aq)+2e⁻→H₂(g) EQ. 2

In other embodiments, the actuation system is configured to drive the plug via positive displacement effectuated by the enlargement of a component within the actuation system, for example, a swellable material (such as a swellable gel) or an enlargeable repository. For example, the actuation system may include one or more of the actuation mechanisms as described in U.S. patent application Ser. No. 13/629,184, entitled “Drug Reconstitution and Delivery Device and Methods,” which is filed concurrently herewith and the disclosure of which is incorporated herein by reference in its entirety. In some embodiments, the drug formulations are dispensed by osmotic swelling displacement. Optionally, a valve may be provided to selectively control the ingress of water into the repository or swellable material. Water from the lumen may be drawn into a repository or swellable material, causing the repository or swellable material to expand in volume. The expansion of the repository or swellable material may displace the drug formulations contained within the reservoir, causing the drug formulations to be dispensed from the device into the lumen. The actuation of the valve may be controlled by the microcontroller.

In other embodiments, the drug formulations are dispensed by an expansive force supplied by an induced phase transformation. For example, the actuation system may include an expandable repository containing a phase-transformable material. The phase-transformable material may be any liquid or solid that will undergo a phase transition from solid or liquid to gas when heated or subjected to an electro-magnetic field. When the material transforms to a gas, the material expands and advances through the reservoir to dispense the drug formulations from the device. The actuation of the phase-transformation may be controlled by the microcontroller.

In other embodiments, the drug formulations are positively displaced and dispensed from the housing by electrostatically-induced compression or using a piezoelectric actuator. For example, a dielectric elastomeric actuator or piezoelectric actuator may be arranged such that a change in voltage or current to the actuator causes the actuator to exert a compressive force on the drug formulations in the reservoir. This compressive force may cause the drug formulations to be dispensed from the device. The actuation of the actuator may be controlled by the microcontroller.

In other embodiments, positive displacement of the drug formulations is achieved using a static pressure head and an actuatable valve. The valve may be operated, for example, in an analog mode for amplitude-modulated dosing or it may be operated in a digital mode for frequency/duty-cycle modulated dosing. The static head pressure may be provided by loading the drug formulations into the device under pressure or the device may be pressurized after the drug formulations are loaded in the device.

In other embodiments, positive displacement of the drug formulations is achieved by mechanical displacement. For example, the mechanical displacement may involve a piston or a spring.

In certain embodiments, the actuation system further includes a wireless receiver for receiving wireless control signals from a separate, detached transmitting device. The device may be deployed into the lumen by the patient, physician, veterinarian, or the like, and thereafter, the patient, physician, veterinarian, or the like, may actuate the release of the drug formulations using the transmitting device to transmit control signals to the deployed device. Furthermore, in some embodiments, the receiver and transmitting device may both be transceivers capable of transmitting and receiving control signals and other communications from each other. Accordingly, in certain embodiments, the transceiver may transmit data relevant to the operation of the device, such as data regarding the drug formulations already administered, the release schedule, the amount of drug formulations remaining in the reservoir, and the remaining battery charge, as well as data relevant to the environment of the lumen, such as data detected or measured by an integral sensor. In some embodiments, the actuation system may also be wirelessly powered.

In certain embodiments, the device is configured for wireless operation, e.g., following deployment in the human or animal subject. In such cases, the device includes appropriate telemetry components as known in the art. For example, actuation of the drug formulation dispensing may be done from a remote controller, e.g., external to the human or animal subject. Generally, the telemetry (i.e. the transmitting and receiving) is accomplished using a first coil to inductively couple electromagnetic energy to a matching/corresponding second coil. The means of doing this are well established, with various modulation schemes such as amplitude or frequency modulation used to transmit the data on a carrier frequency. The choice of the carrier frequency and modulation scheme will depend on the location of the device and the bandwidth required, among other factors. Other data telemetry systems known in the art also may be used. In another case, the device is configured to be remotely powered, or charged. For example, the device may include a transducer for receiving energy wirelessly transmitted to the device, circuitry for directing or converting the received power into a form that can be used or stored, and if stored, a storage device, such as a rechargeable battery or capacitor. In still another case, the device is both wirelessly powered and wirelessly controlled.

In some embodiments, the actuation system may further include one or more sensors for analyzing the environment around the device or within the lumen. For example, a sensor may be employed to detect the temperature or the presence of a drug-degrading enzyme in the lumen. In such embodiments, the microcontroller may be further configured to dispense the drug formulations after the abatement of the drug-degrading enzyme is detected or other suitable environmental conditions are detected for drug delivery.

### Drug Formulations

One or more drug formulations are contained within the device reservoir for delivery to the mucosal tissue. In one embodiment, three drug formulations are disposed within the device reservoir for release to a subject. In another embodiment, as shown in FIG. 3, four drug formulations 318, 320, 332, 334 are disposed within the device reservoir 314 for release to a subject.

The drug formulations may be disposed in the reservoir in a stacked, overlapped, or other configuration. The configuration of the drug formulations within the reservoir may be determined based on the temporal release profile desired. For example, as shown in FIG. 1, the first drug formulation 118 may be provided wholly between the release end of the reservoir and the first barrier 122, with the second drug formulation 120 provided wholly between the first barrier 122 and the plug 124, such that upon actuation, the plug 124 drives the first drug formulation 118 completely out of the reservoir 114 before any of the second drug formulation 120 is released from the reservoir 114. That is, upon actuation, release of the first drug formulation 118 from the reservoir 114 is completed before any of the second drug formulation 120 is released from the reservoir 114.

In another embodiment, as shown in FIG. 3, the first and second drug formulations 318, 320 are separated by a first barrier 322 such that the first and second drug formulations 318, 320 overlap in a direction perpendicular to the actuation axis, and such that upon actuation, the first drug formulation 318 is partially released from the reservoir 314 when the second drug formulation 320 is released from the reservoir 314. That is, upon actuation, release of the first drug formulation 318 from the reservoir 314 overlaps with release of the second drug formulation 320 from the reservoir 314. For example, the first drug formulation 318 may be released only from a first outlet 316 in an end cap at the release end, while the second drug formulation 320 is released only from a second outlet 338 in the end cap. In one embodiment, a third drug formulation 332 is contained in the reservoir such that upon actuation, the second drug formulation 320 is completely released from the reservoir 314 before the third drug formulation 332 is released from the reservoir 314.

Various drug formulations may be administered from the drug delivery device. The different drug formulations within the reservoir may each include the same drug, may each include different drugs, or may be some combination of more than one similar drug and more than one different drug. For example, the first drug formulation may include a different drug than the second drug formulation. For example, the first and third drug formulations may both include the same drug, and second drug formulations may include a different drug than the first and third drug formulations.

In certain embodiments, the device may be used to deliver a battery of drug formulations for a combination therapy, prophylaxis, or for another specific treatment, such as may be useful in animal husbandry.

In one embodiment, the device is used to deliver a fixed time artificial insemination treatment to a human or animal subject. In certain embodiments, the first drug formulation includes a gonadotropin-releasing hormone, the second drug formulation includes a prostaglandin, and the third drug formulation includes a gonadotropin-releasing hormone. In one embodiment, the device also includes a fourth drug formulation which includes a progestin. Variations of the drugs and sequences are envisioned.

In embodiments, the drug formulations include one or more proteins or peptides. In some embodiments, the drug delivery device may be used to administer hormones or steroids, including, but not limited to, follicle stimulating hormone, parathyroid hormone, luteinizing hormone, gonadotropin-releasing hormone (GnRH), estradiol, progesterone, melatonin, serotonin, thyroxine, triiodothyronine, epinephrine, norepinephrine, dopamine, antimullerian hormone, adiponectin, adrenocorticotropic hormone, angiotensinogen, angiotensin, antidiuretic hormone, atrial-natriuretic peptide, calcitonin, cholecystokinin, corticotropin-releasing hormone, erythropoietin, gastrin, ghrelin, glucagon, growth hormone-releasing hormone, human chorionic gonadotropin, human placental lactogen, growth hormone, inhibin, insulin, insulin-like growth factor, leptin, melanocyte stimulating hormone, orexin, oxytocin, prolactin, relaxin, secretin, somatostatin, thrombopoietin, thyroid-stimulating hormone, thyrotropin-releasing hormone, cortisol, aldosterone, testosterone, dehydroepiandrosterone, androstenedione, dihydrotestosterone, estrone, estriol, calcitriol, calcidiol, prostaglandins, leukotrienes, prostacyclin, thromboxane, prolactin releasing hormone, lipotropin, brain natriuretic peptide, neuropeptide Y, histamine, endothelin, enkephalin, renin, and pancreatic polypeptide.

In some embodiments, the drug delivery device may be used to administer cytokine signaling molecules or immunomodulating agents that are used in cellular communication. These molecules commonly comprise proteins, peptides, or glycoproteins. Cytokine signaling molecules include, for example, the four a-helix bundle family which include the IL-2 subfamily (e.g., erythropoietin (EPO) and thrombopoietin (THPO)), the interferon (IFN) subfamily and the IL-10 subfamily. Cytokine signaling molecules also include the IL-1, IL-18, and IL-17 families.

In some embodiments, the drug delivery device may be used to administer drug formulations for pain management, including, but not limited to, corticosteroids, opioids, antidepressants, anticonvulsants (antiseizure medications), non-steroidal anti-inflammatory drugs, COX2 inhibitors (e.g., rofecoxib and celecoxib), ticyclic antidepressants (e.g., amitriptyline), carbamazepine, gabapentin and pregabalin, codeine, oxycodone, hydrocodone, diamorphine, and pethidine.

In some embodiments, the drug delivery device may be used to administer cardiovascular drug formulations. Examples include B-type natriuretic peptide (BNP), atrial natriuretic peptide (ANP), atrial natriuretic factor (ANF), atrial natriuretic hormone (ANH), and atriopeptin. Cardiovascular drug formulations that may be administered by the device also include, for example, antiarrhythmic agents, such as Type I (sodium channel blockers), including quinidine, lidocaine, phenytoin, propafenone; Type II (beta blockers), including metoprolol; Type III (potassium channel blockers), including amiodarone, dofetilide, sotalol; Type IV (slow calcium channel blockers), including diltiazem, verapamil; Type V (cardiac glycosides), including adenosine and digoxin. Other cardiacvascular drug formulations that may be administered by the device include ACE inhibitors, such as, for example, captopril, enalapril, perindopril, ramipril; angiotensin II receptor antagonists, such as, for example, candesartan, eprosartan, irbesartan, losartan, telmisartan, valsartan; beta blocker; and calcium channel blocker.

The drug formulations may be formulated with one or more pharmaceutically acceptable excipients as needed to facilitate the drug's storage in and release from the device. In one embodiment, the drug may be in a liquid solution or suspension. The drug may be in the form of microparticles or nanoparticles. The solvent or carrier may be aqueous or organic. For example, the devices and methods described herein may further include a reconstitution mechanism as described in U.S. patent application Ser. No. 13/629,184, entitled “Drug Reconstitution and Delivery Device and Methods,” which is filed concurrently herewith and the disclosure of which is incorporated herein by reference in its entirety.

In some embodiments, the drug formulations may include components that are degradable by the enzymes present in the fluid secreted by the mucosal tissue. For example, certain amino acids present in drug formulations may be degraded by the enzymes present in fluid secreted by the mucosal tissue. Accordingly, the devices and methods described herein may further include one or more of the permeation enhancement mechanisms described in U.S. Patent Application Publications No. 2011/0087195, No. 2011/0087192, and No. 2011/0087155, the disclosures of which are incorporated herein by reference in pertinent part.

### Methods

Methods are provided for transmucosal drug delivery using intralumenal devices. The intralumenal devices may include any of the device features described herein. The methods include deploying a drug delivery device into the mucosal lumen of a human or animal subject. For example, the subject may be a mammalian animal (e.g., cow, sheep, horse, pig, or dog). The methods include various medical and veterinary therapies, as well as animal husbandry applications. The lumen may be, for example, a vagina, cervix, uterus, bladder, or rectum. The device may be adapted to contact essentially any mucosal tissue surface. The device may be placed in the lumen by inserting the device through an exterior orifice of the patient into the lumen. In some embodiments, the device may be in a form that may be orally administered for delivery of a drug via the mucosal tissue of the gastrointestinal tract.

The drug delivery device includes a reservoir containing first and second drug formulations. In one embodiment, the first and second drug formulations are separated by a first barrier. In another embodiment, the first and second drug formulations are adjacent to each other and are immiscible. The first barrier may be a fluid or solid barrier. For example, the first barrier may include an inert gel.

After the drug delivery device is placed in the mucosal lumen, an actuation system is actuated to drive the first drug formulation out of the reservoir. Thereafter, the actuation system is actuated to drive the second drug formulation out of the reservoir. The first drug formulation may be completely or partially released before the release of the second drug formulation.

As illustrated in FIG. 1, the drug delivery device 100 may be placed in a lumen 152. The drug delivery device may be held in place by frictional engagement between the mucosal tissue and the housing. As shown in FIG. 5A, arms 550 may be provided to facilitate retention of the device within the mucosal lumen. The first and second drug formulations may then be diffused from the at least one outlet in the release end of the reservoir through the porous sidewall membrane via actuation of the actuation system. The actuation of the actuation system may be controlled by the microcontroller. The device may thereafter be removed from the lumen.

A microcontroller may actuate the delivery of the drug formulations by applying an electrical potential to the cathode and the anode of an electrolytic cell. As illustrated in FIGS. 4B-4F, as gas 448 is generated by the electrolytic cell 450 of actuation system 426, the plug 424 advances through the reservoir 414, causing the first and second drug formulations 418, 420 to be driven out of the reservoir 414. The device may thereafter be removed from the lumen.

In another aspect, a method of fixed time artificial insemination is provided. The method may include (i) deploying a drug delivery device having a reservoir containing first, second, and third drug formulations into a vaginal lumen of an animal subject, (ii) actuating an actuation system operable to drive the drug formulations out of the reservoir, (iii) releasing from the reservoir the first drug formulation at a first time, (iv) releasing from the reservoir the second drug formulation at a second time, (v) releasing from the reservoir the third drug formulation at a third time, and (vi) artificially inseminating the animal subject at a fourth time following the first, second, and third times. In one embodiment, the first drug formulation includes a gonadotropin-releasing hormone, the second drug formulation includes a prostaglandin, and the third drug formulation includes a gonadotropin-releasing hormone.

The drug delivery devices may include any of the device features described herein. For example, the device may include a microcontroller configured to control the actuation system, and thereby control the timing of the release of the drug formulations.

In certain embodiments, the method of fixed time artificial insemination further includes releasing from the reservoir the fourth drug formulation including a progestin at a fifth time either before the first time or between the first and second times. In one embodiment, the first time is a time after deployment of the drug delivery device, the second time is from about 5 days to about 7 days after the first time, the third time is from about 2 days to about 3 days after the second time, and the fourth time is either coincident with the third time or from about 8 hours to about 16 hours after the third time.

### Applications/Uses

The drug delivery devices and methods may be used for various medical and therapeutic applications in human and animal subjects.

In some embodiments, the drug delivery device may be used to treat infertility or provide a fixed time artificial insemination (FTAI) treatment in a female subject. For example, the drug delivery device may be placed in the vagina (or uterus, or other part of the birth canal) of a female subject. The drug delivery device may then deliver follicle stimulating hormone to induce ovulation in the female subject. In some embodiments, the drug delivery device may be configured to deliver a plurality of hormones, including follicle stimulating hormone, luteinizing hormone, gonadotropin-releasing hormone separately, or in combination, in appropriate sequences, at appropriate times, and in pharmacologically appropriate amounts. The device may also dispense estradiol to regulate natural hormone production in the female subject. The appropriate dosing schedule and amounts may be determined by one in the field of reproductive pharmacology.

Compared to traditional FTAI treatments, the methods described herein require only device implantation and removal at the time of artificial insemination, and result in a 50% reduction in time spent driving, herding and chuting cattle. The methods also result in improved ovulation quality and quantity due to the reduction in handling, stress, and systemic cortisol levels of the subjects. The methods also reduce the number of medical supplies needed, as a single device delivery the series of FTAI drugs.

In another embodiment, the drug delivery device may be used to treat insulin dependent diabetes (Type I diabetes) in a subject. The drug delivery device may be placed within a lumen of the subject. The drug delivery device may then deliver insulin (Humulin R, Novolin R), insulin isophane (Humulin N, Novolin N), insulin lispro (Humalog), insulin aspart (NovoLog), insulin glargine (Lantus) or insulin detemir (Levemir) to the patient at a selected time or times.

In another embodiment, the drug delivery device may be used to treat diabetes mellitus (Type II diabetes) in a subject. The drug delivery device may be placed within a lumen of the subject. The drug delivery device may then deliver exenatide to the patient at a selected time or times.

In another embodiment, the drug delivery device may be used to treat breast or ovarian cancer in a subject. The drug delivery device may be placed within a lumen of the subject, such as the vagina for a female subject. The drug delivery device may then deliver abraxane (or other drug effective in the treatment or management of cancer) to the patient at a selected time or times.

In another embodiment, the drug delivery device may be used to treat HIV/AIDS in a subject. The drug delivery device may be placed within a lumen of the subject. The drug delivery device may then deliver Abacavir (ABC) or Cidofovir (or other drug effective in the treatment or management of HIV/AIDS) to the patient at a selected time or times. The device also may be used to treat other sexually transmitted diseases.

In another embodiment, the drug delivery device may be used to treat genital herpes in a subject. The drug delivery device may be placed within a lumen of the subject, such as within the vagina of a female subject. The drug delivery device may then deliver acyclovir, famciclovir, or valacyclovir (or other drug effective in the treatment or management of genital herpes) to the patient at a selected time or times.

In another embodiment, the drug delivery device may be used to treat diabetes insipidus in a subject. The drug delivery device may be placed within a lumen of the subject. The drug delivery device may then deliver desmopressin (or other drug effective in the treatment or management of diabetes insipidus) to the patient at a selected time or times.

In another embodiment, the drug delivery device may be used to treat osteoporosis in a subject. The drug delivery device may be placed within a lumen of the subject, such as within the vagina of a female subject. The drug delivery device may then deliver ibandronate, calcitonin, or parathyroid hormone (or other drug effective in the treatment or management of osteoporosis) to the patient at a selected time or times.

It will be appreciated that various of the above-disclosed and other features and functions, or alternatives thereof, may be desirably combined into many other different devices, methods, or applications. Various presently unforeseen or unanticipated alternatives, modifications, variations, or improvements therein may be subsequently made by those skilled in the art which are also intended to be encompassed by the following claims.

1-15. (canceled)

16. A device for drug delivery comprising:
a housing configured for intraluminal deployment into a human or animal subject;
a reservoir contained within the housing and having an actuation end and a release end, the release end comprising at least one outlet;
a first drug formulation disposed within the reservoir;
a second drug formulation disposed within the reservoir and separated from the first drug formulation by a first barrier;
a plug at the actuation end within the reservoir, the plug being movable toward the release end to drive the first and second drug formulations out of the reservoir;
an actuation system operably connected to the actuation end of the reservoir and configured to drive the plug toward the release end such that the first drug formulation is released from the reservoir before the second drug formulation is released from the reservoir; and
a barrier retention chamber connected to the release end of the reservoir and configured to receive and retain the first barrier before the release of the second drug formulation.

17. The device of claim 16, wherein the first barrier comprises an inert gel.

18. The device of claim 16, wherein the first barrier comprises a solid barrier.

19. The device of claim 16, wherein the reservoir is defined by an inner surface of an elongated annular tube.

20. The device of claim 16, wherein the release end of the reservoir comprises a ring of outlets.

21. The device of claim 16, wherein the housing comprises a porous membrane sidewall in fluid communication with the at least one outlet of the release end of the reservoir, the porous membrane sidewall being configured to control diffusion of the first and second drug formulations released from the reservoir.

22. The device of claim 21, wherein the porous membrane sidewall comprises a polypropylene membrane.

23. The device of claim 21, wherein the porous sidewall membrane has a pore size from about 0.2 μm to about 25 μm.

24. The device of claim 16, wherein the barrier retention chamber comprises a hydrophobic vent

25-31. (canceled)
